(19) United States
(12) Patent Application Publication
(10) Pub. No.: US 2017/0015632 A1
(43) Pub. Date: Jan. 19, 2017

(54) METHOD FOR PRODUCING CARBOXAMIDES

(71) Applicant: BAYER CROPSCIENCE AKTIENGESELLSCHAFT, Duesseldorf (DE)

(72) Inventor: SERGII PAZENOK, SOLINGEN (DE)

(21) Appl. No.: 15/300,805

(22) PCT Filed: Mar. 30, 2015
(86) PCT No.: PCT/EP2015/056845

§ 371 Date: Sep. 30, 2016

(30) Foreign Application Priority Data
Apr. 1, 2014 (EP) 14162986.5

(51) Int. Cl. C07D 231/14

(52) U.S. Cl. CPC C07D 231/14

(57) ABSTRACT

The present invention relates to a process for the preparation of carboxamide derivatives of formula (I) as described herein starting from a pyrazole carbonyl derivative and an amine in absence of an additional acid acceptor.

The present invention relates to a novel process for preparing known insecticidally and acaricidally active halogen-substituted compounds from the corresponding pyrazolic acid derivatives (such as halides) and derivatives of aromatic amines in the absence of an acid acceptor.

It is known from the International patent application WO 2010/051926 that pyrazole-carboxamide derivatives can be obtained by reacting the corresponding acid derivatives with the desired aromatic amine derivatives. However, the reaction was carried out in the presence of an activator and in the presence of an acid acceptor.

It is known from the International patent application WO2006/136287 that 5-fluoro-1,3-dimethyl-1H-pyrazole-4-carbonyl fluoride can react in the absence of acid acceptors with different aniline derivatives to form the corresponding carboxyamides. The reaction was possible without any acid acceptors because the week acid (HF) formed during the reaction did not produce salts with low basic anilines and the reaction can go to completion.

Furthermore, it is known from International patent application WO 2006/092291 that 1,3-dimethyl-5-fluoro-4-pyrazolecarbonyl bromide or 1,3-dimethyl-5-fluoro-4-pyrazolecarbonyl chloride, respectively, can react in the absence of acid acceptors with 2-aminoacetophenon. However, 2-aminoacetophenon is a week base and strong acids such as HCl formed during the reaction does not bind the amin completely during the course of the reaction and, thus, do not prevent formation of the amide.

It is not foreseen whether the reaction with other-more basic amines is possible and what reactions conditions are needed especially if reactive fluorocontaining acid chlorides and amines will be reacted without any base. Especially at elevated temperature needed for this coupling a double acetylation could also occur and significantly change the selectivity of the reaction.

Nevertheless, the utilization of the axillary bases (acid acceptors) like NEt₃, Py or inorganic bases like NaOH is usually mandatory for the formation of amides obtained from acid halides and amines (Schotten-Baum process). In many cases the excess of the amines, used for the formation of the amide, can be used as well for trapping of the formed acid. The utilization of the additional base make the process more expensive.

It has now been found that compounds of formula (I)

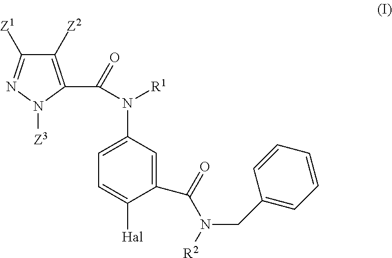

wherein
R¹ and R² are independently selected from hydrogen, optionally halo-substituted C₁-C₄-alkyl, or optionally halo-substituted C₃-C₇-cycloalkyl, preferably hydrogen or C₁-C₂-alkyl, more preferably hydrogen or methyl, even more preferably hydrogen;
Z¹, Z² and Z³ are independently selected from the group consisting of hydrogen, C₁-C₄-alkyl, halo-substituted C₁-C₄-alkyl, C₃-C₆-cycloalkyl, halo-substituted C₃-C₆-cycloalkyl, preferably Z¹ and Z² are independently selected from halo-substituted C₁-C₄-alkyl and Z³ is C₁-C₄-alkyl, more preferably Z¹ and Z² are independently selected from halo-substituted C₁-C₂-alkyl and Z³ is C₁-C₂-alkyl, e.g. Z¹ is pentafluoroethyl, Z² is trifluoromethyl and Z³ is methyl;
Hal is selected from F, Cl, Br or I, preferably Cl,
   can be prepared by reacting a compound of formula (II), a Z¹—Z²—Z³-1H-pyrazole-5-C(═O)-leavinggroup derivative, e.g. Z¹—Z²—Z³-1H-pyrazole-5-carbonyl halide, of the formula (II)

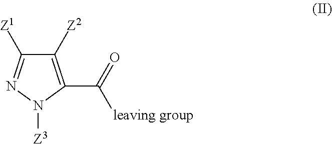

wherein

Z¹, Z², Z³ are as defined as defined herein, and

the leaving group is a halogen selected from the group consisting of F, Cl, Br or I, preferably Cl, with amines derivatives of the formula (III)

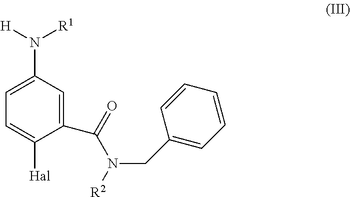

in which R¹, R² and Hal are as defined herein,

in the absence of an acid acceptor (also called acid binder).

In the context of the invention, “in the absence of an acid acceptor” means in the absence of an acid acceptor other than the amine reactant (III) or, in other words, “in the absence of an additional acid acceptor wherein “additional” means in addition to the amine derivative of the formula (III) (or its salts (III′) which is part of the reaction. An “additional acid acceptor” in the sense of the present invention can be a base in addition to the amine compound according to the invention or compounds which reduce the strength of a formed acid such as salts, e.g. silvercyanide (AgCN), which are able to transform strong acids which are formed during the reaction (leaving group anion plus hydrogen cation) into insoluble salts and weak acids (e.g. formed HCl (if the leaving group is chlorine) reacts with AgCN to insoluble AgCl and weak base HCN).

In the context of the present invention, a “haloalkyl” or “halo-substituted alkyl” (which can be used interchangeably) is an alkyl residue wherein at least 1 hydrogen is substituted by a halogen (Halo). In one preferred embodiment, all hydrogen of an alkyl residue are substituted by halogen, e.g. —C(Halo)₃, —C₂(Halo)₅, —C₃(Halo)₇, —C₄(Halo)₉. Further embodiments refer to haloalkyls wherein at least 1, 2, 3, 4, 5, 6, 7, or 8 hydrogens are substituted by halogen. Preferably, a halogen in a haloalkyl is selected from F, Cl, Br or I, more preferably F or Cl, most preferably F. The same definitions and embodiments refer to “halocycloalkyl” or “halo-substituted cycloalkyl”, as well.

Amines can be used also in the form of their salts of the formula (III′)

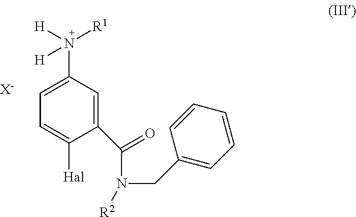

wherein
X is selected from the group consisting of F⁻, Cl⁻, Br⁻, I⁻, HSO₄⁻, CH₃COO⁻, CH₃SO₃⁻, p-Toluolsulphonate, CF₃COO⁻ or CF₃SO₃⁻; preferably Cl⁻, Br⁻, HSO₄⁻, CH₃COO⁻, CH₃SO₃⁻, p-Toluensulphonate, CF₃COO⁻ or CF₃SO₃⁻, and
R¹, R² and Hal are as defined herein.

In one preferred embodiment, the compound of formula (II) is a compound of formula (IIa):

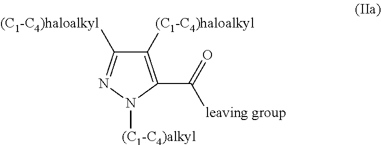

wherein halo is selected from the group consisting of F, Cl, Br or I preferably F and the leaving group is as defined herein.

Preferably, the halo-substituted alkyl-substituents in formula (IIa) are perfluorinated substituents.

In a more preferred embodiment, the compound of formula (II) is the compound of formula (IIb):

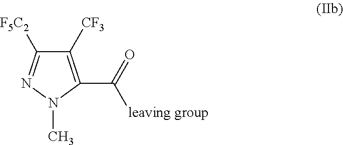

wherein the leaving group is as defined herein.

In one more preferred embodiment, the leaving group is Cl or F, even more preferred Cl (compound (IIc):

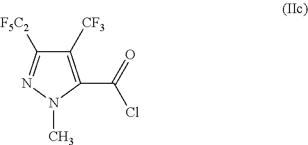

In another preferred embodiment, an amine derivative of the formula (III) is a compound of formula (IIIa) or its salt (IIIa′):

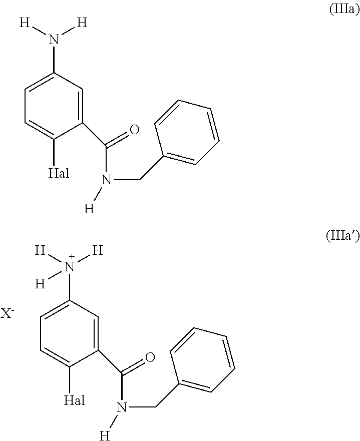

wherein
Hal is selected from F, Cl or Br, preferably, Hal is Cl; and
X⁻ (in the case of compound (IIIa′) is selected from the group consisting of F⁻, Cl⁻, Br⁻, I⁻, HSO₄⁻, CH₃COO⁻, BF₄⁻, CH₃SO₃⁻, Toluensulphonate (anionic form of Toluensulphonic acid), CF₃COO⁻ or CF₃SO₃⁻.

Surprisingly, the carboxamides of the formula (I) can be prepared under the inventive conditions with good yields in high purity and selectivity. A further advantage of the process according to the invention is that the workup is simpler, since an acid acceptor is not needed. This causes fewer or no waste water, an easier purification process without prior isolation by addition of an aliphatic alcohol in the same reaction vessel, and the process can be run in a higher concentration. The resulting product has then been obtained with a surprising purity superior to 90% or even close to 100%, and with less reagent and effort, while prior conditions in presence of an acid acceptor generally leads to a purity close to less than 90% The process according to the invention becomes more economically viable.

One preferred embodiment refers to a reaction for the production of compounds of formula (Ia)

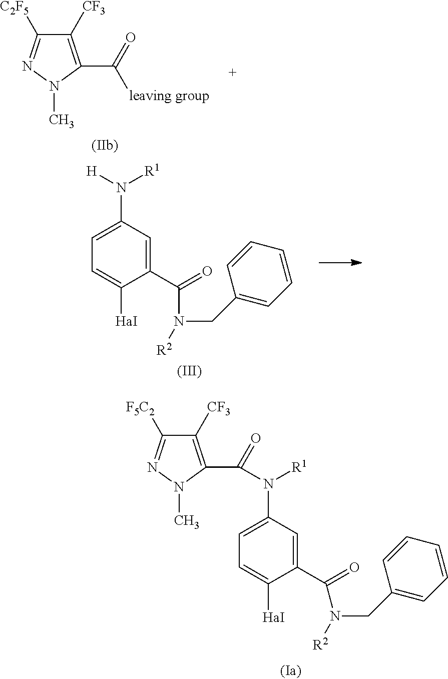

wherein leaving group refers to F, Cl, Br or I, preferably F or Cl, and
R¹ and R² are independently selected from hydrogen, optionally halo-substituted C₁-C₄-alkyl, or optionally halo-substituted C₃-C₇-cycloalkyl, preferably hydrogen or C₁-C₂-alkyl, more preferably hydrogen or methyl, even more preferred hydrogen;
Hal is selected from F, Cl, Br or I, preferably F or Cl, more preferably Cl,
   in the absence of a in the absence of an acid acceptor in addition to compound

The same process can be carried out when using compound (III′) instead of compound (III).

Another preferred embodiment refers to a reaction for the production of compounds of formula (Ia)

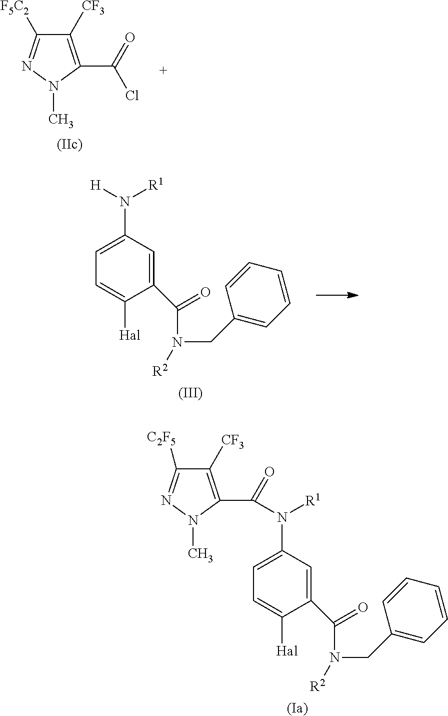

wherein
R¹ and R² are independently selected from hydrogen, optionally halo-substituted C₁-C₄-alkyl, or optionally halo-substituted C₃-C₇-cycloalkyl, preferably hydrogen or C₁-C₂-alkyl, more preferably hydrogen or methyl, even more preferred hydrogen; and
Hal is selected from F, Cl, Br or I, preferably F or Cl, more preferably Cl,
   in the absence of a in the absence of an acid acceptor in addition to compound (III).

The same process can be carried out when using compound (III′) instead of compound (III).

When, for example, 1-methyl-3-pentafluorethyl-4-tifluoromethyl-1H-pyrazole-5-carbonyl chloride and 5-amino-N-benzyl-2-chlorobenzamide are used as starting materials, the process according to the invention can be illustrated by the following formula scheme:

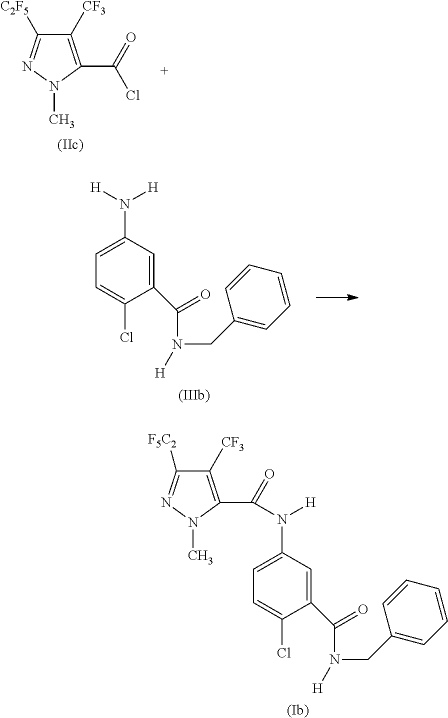

in the absence of a in the absence of an acid acceptor in addition to compound
A salt of compound (IIIb) as defined herein can be used, a well to carry out this reaction.

The same process can be carried out when using compound (IIIb′)

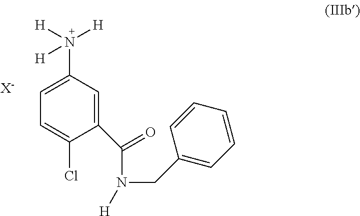

(wherein X⁻ is as defined herein) instead of compound (IIIb).

The carbonyl chloride and fluoride of the formula (II) (and the production thereof) used as a starting material in the performance of the process according to the invention are known from, e.g. WO 2010/051926.

Preference is given in the use of amines of the formula (IIIb) or its salt (IIIb′).

The process according to the invention is preferably used to prepare compound of formula (Ib):

N-[3-(benzylcarbamoyl)-4-chlorophenyl]-1-methyl-3-(pentafluoroethyl)-4-(trifluoromethyl)-1H-pyrazole-5-carboxamide.

One aspect also refers to the use of a compound of formula (II), preferably of formula (IIa), (IIb) or (IIc) for preparing a compound of formula (I).

Use of compound of formula (III), preferably of formula (IIIa) or (IIIb) or a salt thereof for preparing a compound of formula (I).

Amines derivatives of the formula (III) and their salts are known or can be prepared in a known manner (see, e.g., WO 2010/051926). The process according to the invention can be performed in the presence of a diluent. Useful diluents for this purpose include all inert organic solvents, preferably aliphatic, alicyclic or aromatic hydrocarbons, for example petroleum ether, hexane, heptane, cyclohexane, methylcyclohexane, benzene, toluene, xylene or decalin; halogenated hydrocarbons, for example chlorobenzene, dichlorobenzene, dichloromethane, chloroform, tetrachloromethane, dichloroethane or trichloroethane; ethers such as diethyl ether, diisopropyl ether, methyl t-butyl ether, methyl t-amyl ether, dioxane, tetrahydrofuran, 1,2-dimethoxyethane, 1,2-diethoxyethane or anisole; ketones such as acetone, butanone, methyl isobutyl ketone or cyclohexanone; nitriles such as acetonitrile, propionitrile, n- or i-butyronitrile or benzonitrile; amides such as N,N-dimethylformamide, N,N-dimethylacetamide, N-methylformanilide, N-methylpyrrolidone or hexamethylphosphoramide, more preferably are used chlorobenzene and toluene.

Preferred diluents are aliphatic, alicyclic or aromatic hydrocarbons, for example petroleum ether, hexane, heptane, cyclohexane, methylcyclohexane, benzene, toluene, xylene or decalin; and halogenated hydrocarbons, for example chlorobenzene, dichlorobenzene, dichloromethane, chloroform, tetrachloromethane, dichloroethane or trichloroethane; e.g. toluene or chlorbenzene.

The reaction temperatures in the performance of the process according to the invention can be varied within a relatively wide range. In general, temperatures of from 70° C. to 150° C., preferably temperatures of from 80° C. to 140° C., e.g. 100° C. or around 100° C. such as 80° C. to 130° C. or 80° C. to 120° C., are employed.

For the process according to the invention, generally between 0.8 and 1.5 mol, preferably 0.8 to 1.4 mol, 0.9 to 1.4 mol, equimolar amounts or 1 to 1.2 mol of amine derivative of the formula (III), preferably (IIIa), (IIIa′), (IIIb) or (IIIb′), especially preferred (IIIb) or (IIIb′), are used per mole of the pyrazole-carboxamide derivatives (II), preferably (IIa) to (IIf). 1-methyl-3-difluoromethyl-5-fluoro-1H-pyrazole-4-carbonyl halides of the formula (II).

One preferred embodiment refers to a reaction of a compound (IIIb) or its salt (IIIb′), respectively, with compound (IIc), wherein the ratio of compound (IIIb) (or its salt (IIIb′)) and (IIc) is between 1:1 to 1:3, preferably between 1:1 to 1:2 such as between 1:1 to 1:1.3 or even 1:1.

Depending on the reactivity of the reactants, the reaction time may be up to 15 hours, but the reaction can also be terminated even earlier in the case of complete conversion. Preference is given to reaction times of 5-10 hours.

All processes according to the invention are generally performed under standard pressure. However, it is possible to work under elevated or reduced pressure generally between 0.1 bar and 10 bar It is preferable to work under reduced pressure to remove HCl from the reaction volume.

All processes according to the invention can generally be performed under atmosphere. However, it is preferred to carry out the processes according to the invention under protective gas such as argon. or nitrogen.

For the work up it is enough to remove the solvent and precipitate the formed product. It is also possible to extract the product and wash the solution with water. In all cases the product was formed in purity more than 95% so any further purification was not needed.

The inventive preparation of carboxamides of the formula (I) is described in the examples which follow, which further illustrate the above description. However, the examples should not be interpreted in a restrictive manner.

### PREPARATION EXAMPLES

### Preparation Example

### N-[3-(benzylcarbamoyl)-4-chlorophenyl]-1-methyl-3-(pentafluoroethyl)-4-(trifluoromethyl)-1H-pyrazole-5-carboxamide

Under protective gas (argon), a solution of 5-amino-N-benzyl-2-chlorobenzamide 26 g (100 mmol) in 100 ml of toluene is initially charged. 33 g (100 mmol) of 1-methyl-3-pentafluorethyl-4-tifluoromethyl-1H-pyrazole-5-carbonyl chloride are added and the mixture is stirred at 100° C. for 8 h. For workup 50 ml of the solvent was removed in vacuum and the precipitate was filtered off to give 53.6 g with a purity w.w. % 96-97 (93% of theory %) N-[3-(benzylcarbamoyl)-4-chlorophenyl]-1-methyl-3-(pentafluoroethyl)-4-(trifluoromethyl)-1H-pyrazole-5-carboxamide in the form of white crystals with melting point of 174° C.

Yield was calculated as usually for 100% pure compound. It means 53.6 g with a purity of 96-97 w.w. % gave 51.45 pure (100%) compound, or 93% yield of the pure compound (51.45:55.4 theory).

### Preparation Example

### N-[3-(benzylcarbamoyl)-4-chlorophenyl]-1-methyl-3-(pentafluoroethyl)-4-(trifluoromethyl)-1H-pyrazole-5-carboxamide

Under protective gas (argon), a solution of 5-amino-N-benzyl-2-chlorobenzamide [symbol] 26 g (100 mmol) in 80 ml of chlorobenzene is initially charged. 33 g (100 mmol) of 1-methyl-3-pentafluorethyl-4-tifluoromethyl-1H-pyrazole-5-carbonyl chloride are added and the mixture is stirred at 105° C. for 8 h. For workup 45 ml. of the solvent was removed in vacuum and the precipitate was filtered off to give 55 g N-[3-(benzylcarbamoyl)-4-chlorophenyl]-1-methyl-3-(pentafluoroethyl)-4-(trifluoromethyl)-1H-pyrazole-5-carboxamide in the form of white crystals with melting point of 172-174° C. and purity w.w. % 96%, yield 95%.

Yield was calculated as usually for 100% pure compound. It means 55 g N-[3-(benzylcarbamoyl)-4-chlorophenyl]-1-methyl-3-(pentafluoroethyl)-4-(trifluoromethyl)-1H-pyrazole-5-carboxamide with a purity of 96 w.w. % gave 52.8 g pure (100%) compound, or yield 95% yield of the pure compound 52.8 g: 55.4 g=95% of the theory.

1. A process for the preparation of one or more carboxamide derivatives of formula (I) 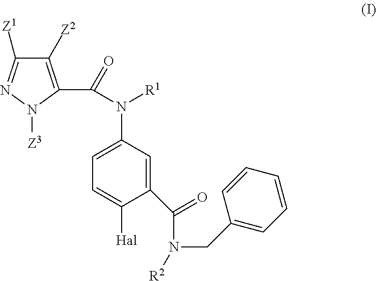
wherein
R¹ and R² are independently selected from hydrogen, optionally halo-substituted C₁-C₄-alkyl, or optionally halo-substituted C₃-C₇-cycloalkyl, optionally hydrogen or C₁-C₂-alkyl, optionally hydrogen or methyl, even more preferably hydrogen;
Z¹, Z² and Z³ are independently selected from the group consisting of hydrogen C₁-C₄-alkyl, halo-substituted C₁-C₄-alkyl, C₃-C₆-cycloalkyl, halo-substituted C₃-C₆-cycloalkyl, optionally Z¹ and Z² are independently selected from halo-substituted C₁-C₄-alkyl and Z³ is C₁-C₄-alkyl, optionally Z¹ and Z² are independently selected from halo-substituted C₁-C₂-alkyl and Z³ is C₁-C₂-alkyl;
Hal is selected from F, Cl, Br or I, optionally Cl,
comprising reacting one or more compounds of the formula (II) 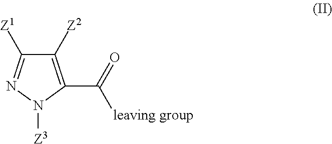
wherein
Z¹, Z² and Z³ are independently selected from the group consisting of hydrogen, C₁-C₄-alkyl, halo-substituted C₁-C₄-alkyl, C₃-C₆-cycloalkyl, halo-substituted C₃-C₆-cycloalkyl, optionally Z¹ and Z² are independently selected from halo-substituted C₁-C₄-alkyl and Z³ is C₁-C₄-alkyl, optionally Z¹ and Z² are independently selected from halo-substituted C₁-C₂-alkyl and Z³ is C₁-C₂-alkyl; and
the leaving group is C₁-C₄-alkoxy, or a halogen selected from F, Cl, Br or I, or
with one or more amine derivatives of formula (III) or a salt thereof (III′) 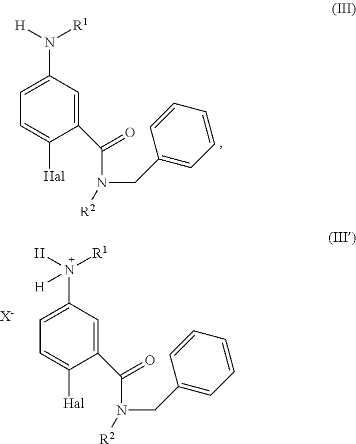
wherein
R¹ and R² are independently selected from hydrogen, optionally halo-substituted C₁-C₄-alkyl, or optionally halo-substituted C₃-C₇-cycloalkyl, optionally hydrogen or C₁-C₂-alkyl, optionally hydrogen or methyl, optionally hydrogen;
Hal is selected from F, Cl, Br or I, optionally preferably Cl
X is selected from the group consisting of F⁻, Cl⁻, Br⁻, I⁻, HSO₄⁻, CH₃COO⁻, BF₄⁻, CH₃SO₃⁻, p-Toluensulphonate, CF₃COO⁻ or CF₃SO₃⁻;
in the absence of an acid acceptor in addition to compound (III) or (IY).

2. A process according to claim 1, wherein the amine derivatives are of formula (IIIa) or a salt thereof (IIIa′) 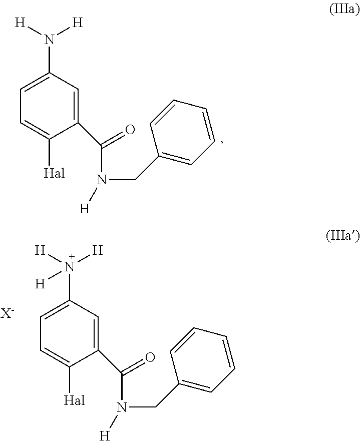
wherein
Hal is selected from F, Cl or Br. optionally, Hal is Cl; and
X⁻ is selected from the group consisting of F⁻, Cl⁻, Br⁻, I⁻, HSO₄⁻, CH₃COO⁻, BF₄⁻, CH₃SO₃⁻, CF₃COO⁻ or CF₃SO₃⁻.

3. A process according to claim 1, wherein the amine derivatives are of formula (IIIb) or a salt thereof (IIIb′) 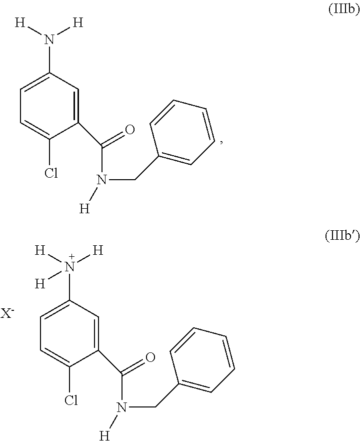
wherein
X⁻ is selected from the group consisting of F⁻, Cl⁻, Br⁻, I⁻, HSO₄⁻, CH₃COO⁻, BF₄⁻, CH₃SO₃⁻, CF₃COO⁻ or CF₃SO₃⁻.

4. A process according to claim 1, wherein a compound of formula (II) is a compound of formula 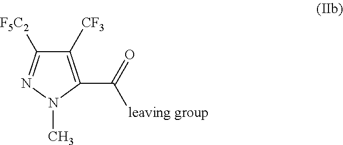
wherein the leaving group is selected from F, Cl, Br or I.

5. A process according to claim 1, wherein the compound of formula (II) is the compound of formula (IIc):

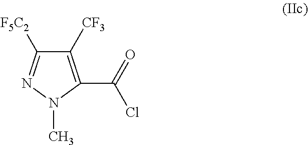

6. A process according to claim 1, wherein the compound of formula (I) is N-[3-(benzylcarbamoyl)-4-chlorophenyl]-1-methyl-3-(pentafluoroethyl)-4-(trifluoromethyl)-1H-pyrazole-5-carboxamide, the compound of formula (II) is compound of formula (IIb) and the compound of formula (III) is compound of formula (IIIb).

7. A process according to claim 1, wherein the ratio of compound (IIIb) (or its salt (IIIb′)) and (IIc) is between 1:1 and 1:3.

8. A method of using a compound of formula (II), optionally of formula (IIa), (IIb) or (IIc) for preparing a compound of formula (I).

9. A method of using compound of formula (III), optionally of formula (IIIa) or (IIIb) or a salt thereof for preparing a compound of formula (I).
